(19) United States
(12) Patent Application Publication
(10) Pub. No.: US 2019/0127781 A1
(43) Pub. Date: May 2, 2019

(54) USE OF A MICROBIOME PROFILE TO DETECT LIVER DISEASE

(71) Applicant: HUMAN LONGEVITY, INC., San Diego, CA (US)

(72) Inventors: Shibu YOOSEPH, San Diego, CA (US); Rohit LOOMBA, San Diego, CA (US); Karen E. NELSON, San Diego, CA (US); Victor SEGURITAN, San Diego, CA (US); Weizhong LI, San Diego, CA (US); Tao LONG, San Diego, CA (US); Niels KLITGORD, San Diego, CA (US)

(21) Appl. No.: 16/095,334

(22) PCT Filed: Apr. 20, 2017
(86) PCT No.: PCT/US2017/028691

§ 371 Date: Oct. 19, 2018

Related U.S. Application Data
(60) Provisional application No. 62/325,334, filed on Apr. 20, 2016.

(51) Int. Cl. C12Q 1/689; C12Q 1/6883; G16H 50/20; G16H 50/30

(52) U.S. Cl. CPC C12Q 1/689; C12Q 1/6883; G16H 50/20; C12Q 2600/118; C12Q 2600/112; C12Q 2600/156; G16H 50/30

(57) ABSTRACT

A method to detect liver fibrosis or for the differential diagnosis of non-alchohlic fatty liver disease (NAFLD) in a subject is provided. The method comprises analyzing a biological sample from a subject to determine an intestinal microbiome signature for the subject, and inspecting the intestinal microbiome signature relative to a reference intestinal microbiome signature to detect presence or absence of liver fibrosis.

### CROSS-REFERENCE TO RELATED APPLICATIONS

This application claims the benefit of U.S. Provisional Application No. 62/325,334, filed Apr. 20, 2016, incorporated herein by reference in its entirety.

### TECHNICAL FIELD

The subject matter described herein relates to methods for detection of liver fibrosis and for the detection and/or differentiation of non-alcoholic liver diseases by analyzing a sample from a subject to determine its microbiome signature for presence, absence, or relative abundance of bacterial species.

### BACKGROUND

The human intestinal microbiota consists of trillions of microorganisms including 150-200 prevalent and 1000 less common bacterial species, harboring over 100-fold more genes than those present in the human genome (Quigley, et al., J. Hepatology, 58:1020-1027 (2013)). The intestinal microbiota is composed predominantly of bacteria, yet also contains archaea, protozoa, and viruses. The microbiota performs vital functions essential to health maintenance, including food processing, digestion of complex indigestible polysaccharides and synthesis of vitamins, and it secretes bioactive metabolites with diverse functions, ranging from inhibition of pathogens, metabolism of toxic compounds to modulation of host metabolism (Quigley, Id.).

A perturbed microbiota has been implicated in various disorders in humans, from necrotizing enterocolitis in infants, to obesity, diabetes, metabolic syndrome, irritable bowel syndrome, and inflammatory bowel disease in adults. Though the role of the microbiota in the pathogenesis of some human disorders is recognized, a firm scientific basis for a role for the gut microbiome in liver disease is still emerging. For example, it has been suspected that the gut microbiota might play a role in the pathogenesis or progression of certain liver diseases, including alcoholic liver disease, non-alcoholic fatty liver disease (NAFLD) and non-alcoholic steato-hepatitis (NASH), total parenteral nutrition/intestinal failure-related liver disease, and primary sclerosing cholangitis (Quigley, supra). A method to readily and accurately assess the microbiota present in an individual and to correlate the presence, absence, or relative abundance of particular microbes with particular diseases and conditions, and/or the risk of developing the same is needed.

With respect to NAFLD, approximately 80-100 million Americans are estimated to have NAFLD, the hepatic manifestation of metabolic syndrome, commonly associated with obesity and insulin resistance (Carding, S. et al., Microb. Ecol. Health Dis., 26:26191 (2015); Zhu, L. et al., Hepatology, 57(2):601-609 (2013); Kakiyama, G. et al., J. Hepatol., 58(5):949-955 (2013)). NAFLD is a spectrum of liver disease ranging from benign steatosis, referred to as nonalcoholic fatty liver (NAFL) that is the non-progressive subtype of NAFLD, to nonalcoholic steatohepatitis (NASH), the progressive subtype of NAFLD, which can progress to cirrhosis, hepatocellular carcinoma and liver-related death (Qin, N., et al., Nature, 513(7516):59-64 (2014)). NAFL and NASH are typically differentiated by a liver biopsy, and an alternative method, preferably a non-invasive method, for detection of these disorders and for their differentiation is desired.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method to detect liver fibrosis in a subject is provided. The method comprises analyzing a biological sample from a subject to determine an intestinal microbiome signature for the subject; and inspecting the intestinal microbiome signature relative to a reference intestinal microbiome signature to detect presence or absence of liver fibrosis.

In another aspect, a method to detect liver fibrosis or for the differential diagnosis of type of non-alcoholic fatty liver disease (NAFLD) in a subject is provided. The method comprises analyzing a biological sample from a subject to determine an intestinal microbiome signature for the subject; and inspecting the intestinal microbiome signature relative to a reference intestinal microbiome signature to detect presence or absence of liver fibrosis; and/or inspecting the intestinal microbiome signature to determine whether at least n bacterial species identified in Table 2 is present or absent in the signature, where n is at least 2, wherein presence or absence of the at least n bacterial species identified in Table 2 in the intestinal microbiome signature indicates nonalcoholic steatohepatitis (NASH).

In one embodiment, analyzing comprising applying the biological sample to a test panel that detects at least n bacterial species identified in Table 2, Table 3, Table 4 and/or Table 5. In one embodiment, n is at least two.

In another embodiment, analyzing further comprises defining an intestinal microbiome signature according to the presence or absence of the at least n bacterial species identified in Table 2, Table 3, Table 4 and/or Table 5.

In other embodiments, the intestinal microbiome signature is compared to a reference intestinal microbiome signature obtained from a population of subjects without liver fibrosis.

In one embodiment, the intestinal microbiome signature is compared to a reference intestinal microbiome signature from obtained from a population of subjects with liver fibrosis.

In another embodiment, the reference intestinal microbiome signature is obtained from a population of subjects with advanced liver fibrosis.

In still other embodiments, a relative abundance of bacterial species in the intestinal microbiome signature is determined from a median abundance of each bacterial species in the intestinal microbiome signature relative to a median abundance of each bacterial species in the reference intestinal microbiome signature.

In yet another embodiment, analyzing a biological sample comprises a sample selected from the group consisting of a stool sample, an intestinal mucosal sample and a sample of the intestinal contents.

In other embodiments, based on the inspecting, a stage of liver fibrosis is determined.

In one embodiment, based on the inspecting, a stage of advanced fibrosis is determined.

In other embodiments, based on the inspecting, a differential diagnosis of the type of non-alcoholic fatty liver disease (NAFLD) is determined.

In some embodiments, n is selected from the group consisting of one (1), two (2), three (3), four (4), five (5), six (6), seven (7), eight (8), nine (9), ten (10), eleven (11), twelve (12), thirteen (13), fourteen (14), fifteen (15), sixteen (16), seventeen (18), nineteen (19) and twenty (20).

In still other embodiments, n is selected from the group consisting of about 1-30, about 1-25, about 5-30, about 5-25, about 10-30, and about 10-25.

In yet other embodiments, n is selected from the group consisting of greater than one (1), greater than two (2), greater than three (3), greater than four (4), greater than five (5), greater than six (6), greater than seven (7), greater than eight (8), greater than nine (9), greater than ten (10), greater than eleven (11), greater than twelve (12), greater than thirteen (13), greater than fourteen (14), greater than fifteen (15), greater than sixteen (16), greater than seventeen (18), greater than nineteen (19) and greater than twenty (20). In one embodiment, n is less than 40 or less than 37 or less than 35 or less than 30.

In one embodiment, n is at least 8 and is comprised of the bacterial species in Group A (Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae.

In another embodiment, the intestinal microbiome signature comprised of the Group A bacterial species have a relative abundance of each bacterial species in the signature at least two-fold higher than a relative abundance of the Group A bacterial species in a reference intestinal microbiome signature.

In another embodiment, n is at least 9 and additionally comprises one or more of the bacterial species in Group B (Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alistipes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG:83, Dorea longicatena, Firmicutes bacterium CAG:129, Ruminococcus obeum CAG:39, Blautia sp. CAG:37, Eubacterium rectale, Firmicutes bacterium CAG:176, Firmicutes bacterium CAG:110, and Holdemania filiformis).

In one embodiment, the Group B bacterial species in the intestinal microbiome signature have a relative abundance at least two-fold lower than a relative abundance of the Group B bacterial species in a reference intestinal microbiome signature.

In another embodiment, n is comprised of the bacterial species in Group C (gathobacter rectalis (Eubacterium rectale), Blautia sp. KLE 1732, Roseburia inulinivorans, Oscillibacter (genus), Eubacterium ramulus, and Blautia sp. GD8).

In yet another embodiment, the Group C bacterial species in the intestinal microbiome signature have a relative abundance at least two-fold lower than a relative abundance of the Group C bacterial species in a reference intestinal microbiome signature.

In some embodiments, analyzing comprises analyzing the biological sample using a microarray comprising nucleic acid sequences with binding affinity for one or more bacterial species set forth in Table 2, Table 3, Table 4 and/or Table 5.

In one embodiment, the nucleic acid is DNA, cDNA, RNA, mRNA, or rRNA.

In another embodiment, analyzing comprises analyzing the biological sample using a nucleic acid amplification technique.

In still another embodiment, the nucleic acid amplification technique is selected from real-time polymerase chain reaction and reverse transcription polymerase chain reaction.

In another embodiment, the nucleic acid amplification technique is an isothermal nucleic acid amplification technique.

In yet another embodiment, analyzing comprises analyzing the biological sample using nucleic acid sequencing.

In another embodiment, the nucleic acid sequencing comprises total DNA sequencing or sequencing of the complete 16SrRNA gene or sequencing of a hypervariable region of the 16S rRNA gene, including but not limited to the V6 region. Next-generation sequencing (NGS) is used in some embodiments to analyze the biological sample.

In one embodiment, the nucleic acid sequencing comprises DNA sequencing by pyrosequencing or Sanger sequencing. The pyrosequencing in one embodiment is multitag sequencing.

In other embodiments, analyzing comprises analyzing using a method selected from microscopy, metabolite identification, Gram staining, flow cytometry, immunological assays, and culture-based assays.

In another aspect, a method for the differential diagnosis of the type of non-alcoholic fatty liver disease (NAFLD) in a subject, comprising determining whether at least n bacterial species identified in Table 2, Table 3, Table 4 and/or Table 5 is present in intestinal microflora of the subject, wherein presence of the at least n bacterial species identified in Table 2, Table 3, Table 4 and/or Table 5 in the intestinal microflora indicates nonalcoholic steatohepatitis (NASH).

In another aspect, a method for the differential diagnosis of the type of non-alcoholic fatty liver disease (NAFLD) in a subject is provided. The method comprises determining an intestinal microbiome signature of the subject, wherein a diagnosis of stage 3-4 fibrosis is indicated by one or more of the following criterion:
(a) the intestinal microbiome signature of the subject having a relative abundance of a bacterial species that is at least two-fold higher than the relative abundance of the bacterial species in a reference intestinal microbiome signature, wherein the bacterial species is selected from the group consisting of Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae (Group A);
(b) the intestinal microbiome signature of the subject having a relative abundance of a bacterial species that is at least two-fold lower than the relative abundance of the bacterial species in a reference intestinal microbiome signature, wherein the bacterial species is selected from the group consisting of Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alistipes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG:83, Dorea longicatena, Firmicutes bacterium CAG: 129, Ruminococcus obeum CAG:39, Blautia sp. CAG:37, Eubacterium rectale, Firmicutes bacterium CAG:176, Firmicutes bacterium CAG:110, and Holdemania filiformis (Group B); and/or
(c) the intestinal microbiome signature of the subject having a relative abundance of a bacterial species that is at least about two-fold lower or at least about 2.5-fold lower than the relative abundance of the bacterial species in a reference intestinal microbiome signature, wherein the bacterial species is selected from the group consisting of Agathobacter rectalis (Eubacterium rectale), Blautia sp. KLE 1732, Roseburia inulinivorans, Oscillibacter (genus), Eubacterium ramulus, and Blautia sp. GD8 (Group C); and/or
(d) the intestinal microbiome signature of the subject having (i) a relative abundance of at least two bacterial species in Table 2 that is at least two-fold higher than the relative abundance of the same two bacterial species in a reference intestinal microbiome signature, (ii) a relative abundance of at least two bacterial species in Table 2 that is at least two-fold lower than the relative abundance of the same two bacterial species in a reference intestinal microbiome signature; and (iii) the sum of the mean decrease in Gini index of the at least two bacterial species in (i) and of the at least two bacterial species in (ii) is greater than and/or greater than or equal to 0.5.

In one embodiment, the diagnosis of stage 3-4 fibrosis is indicated by both (a) and (b), by both (a) and (c), by both (a) and (d), by both (b) and (c), or by both (b) and (d).

In another embodiment, the sum of the mean decrease in Gini index of the at least two bacterial species in (i) and of the at least two bacterial species in (ii) is greater than and/or greater than or equal to 0.7.

In another embodiment, the diagnosis of stage 3-4 fibrosis is indicated by at least two of the bacterial species listed in (a) having a relative species abundance in the subject microbiome signature that is at least two-fold higher than in the reference microbiome signature.

In yet another embodiment, the diagnosis of stage 3-4 fibrosis is indicated by at least two of the bacterial species listed in (b) or in (c) having a relative species abundance in the subject microbiome signature that is at least two-fold lower than in the reference microbiome signature.

In still another embodiment, the diagnosis of stage 3-4 fibrosis is indicated by the intestinal microbiome signature of the subject having a relative abundance of a bacterial species that is at least two-fold lower than the relative abundance of the bacterial species in a reference intestinal microbiome signature, wherein the bacterial species is selected from the group consisting of Oscillibacter_sp._CAG.241, Firmicutes_bacterium CAG.129, Firmicutes_bacterium_CAG.170, Ruminococcus_obeum, Bacteroides_pectinophilus, Holdemania_jiliformis, and Firmicutes_bacterium_CAG. 83.

In another embodiment, the diagnosis of stage 3-4 fibrosis is additionally indicated by an increased abundance of E. coli in the intestinal microbiome signature of the subject relative to the reference microbiome.

In another aspect, a method for the differential diagnosis of the type of non-alcoholic fatty liver disease (NAFLD) in a subject, comprising analyzing intestinal microflora of the subject to determine an intestinal microbiome signature for the subject; and inspecting the intestinal microbiome signature to determine whether at least n bacterial species identified in Table 2, Table 3, Table 4 and/or Table 5 is present in the signature. Presence of the at least n bacterial species identified in Table 2, Table 3, Table 4 and/or Table 5 in the intestinal microbiome signature indicates nonalcoholic steatohepatitis (NASH).

In another aspect, a substantially non-invasive method for assessing risk of progression to liver cirrhosis in a subject having an intestinal microbiome signature and diagnosed with non-alcoholic fatty liver disease (NAFLD), comprising (i) determining whether at least n bacterial species identified in Table 2, Table 3, Table 4 and/or Table 5 is present in the intestinal microbiome signature of the subject, wherein presence of the at least n bacterial species identified in Table 2, Table 3, Table 4 and/or Table 5 in the intestinal microbiome signature indicates risk of progression to liver cirrhosis, or (ii) determining from the intestinal microbiome signature of the subject (i) a relative abundance of at least two bacterial species in Table 2 that is at least two-fold higher than the relative abundance of the same two bacterial species in a reference intestinal microbiome signature and (ii) a relative abundance of at least two bacterial species in Table 2 that is at least two-fold lower than the relative abundance of the same two bacterial species in a reference intestinal microbiome signature, and (iii) a mean decrease in Gini index of the at least two bacterial species in (i) and of the at least two bacterial species in (ii), wherein if the sum of the mean decrease in Gini index is greater than 0.5 risk of progression to liver cirrhosis is indicated.

In one embodiment, n is at least two.

In still another aspect, an assay method to differentiate nonalcoholic fatty liver (NAFL) from nonalcoholic steatohepatitis (NASH) in a subject with non-alcoholic fatty liver disease (NAFLD) is provided. The method comprises analyzing intestinal microflora of the subject to determine an intestinal microbiome signature for the subject; and inspecting the intestinal microbiome signature to determine whether at least n bacterial species identified in Table 2 is present in the signature, wherein presence of the at least n bacterial species identified in Table 2 in the intestinal microbiome signature indicates nonalcoholic steatohepatitis (NASH).

In one embodiment, absence of the at least n bacterial species identified in Table 2, Table 3, Table 4 and/or Table 5 in the intestinal microbiome signature indicates nonalcoholic fatty liver (NAFL).

In another embodiment, n is selected from the group consisting of one (1), two (2), three (3), four (4), five (5), six (6), seven (7), eight (8), nine (9), ten (10), eleven (11), twelve (12), thirteen (13), fourteen (14), fifteen (15), sixteen (16), seventeen (18), nineteen (19) and twenty (20).

In another embodiment, n is selected from the group consisting of 1 and less than 30, 1 and less than 25, 5 and less than 30, 5 and less than 25, 10 and less than 30, and 10 and less than 25.

In still other embodiments, the intestinal microflora are obtained from a biological sample from the subject, the sample selected from the group consisting of a stool sample, an intestinal mucosal sample and a sample of the intestinal contents.

In one embodiment, the bacterial species present in the intestinal microflora are determined using microarray comprising nucleic acid sequences with binding affinity for bacterial species set forth in Table 2, Table 3, Table 4 and/or Table 5.

In another embodiment, the intestinal microbiome signature is based on bacterial metabolic products from intestinal microflora or on proteins in intestinal microflora, and a bacterial microbiome signature is determined from the metabolic products or the proteins.

In another embodiment, determining or inspecting comprises use of a methodology selected from the group consisting of non-parametric multivariate analysis, random forest analysis, a Support Vector Machine, correlation network analysis, correlation difference network analysis, Bayesian models, Linear Models and supervised machine learning tool.

Additional embodiments of the present methods will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are boxplots of the relative abundances of 37 species selected for the random forest model used to distinguish samples in the mild/moderate group (G1, open bars) from those in the advanced fibrosis group (G2, dashed fill).

FIG. 2 is an overview of metabolomic and metagenomic analyses of Biopsy-proven NAFLD patients. Serum and stool samples from a cohort of 86 patients were analyzed for their metabolic and functional content. Left: The metabolic profiles of 56 serum samples detected several differentially abundant metabolites, after multiple test correction. These are denoted by unfilled, open boxes for the G1 enriched dashed-fill boxes for G2 enriched. Center: ORF sequences identified from whole genome sequencing of 86 stool samples were used to compute relative abundances of enzymes involved in SCFA production. Several enzymes were enriched in either G1 (unfilled, open) or G2 (dashed fill), though they were not statistically significant after multiple test correction. Right: Metabolic pathways were reconstructed from whole genome sequencing of 86 stool samples. Pathway abundance was calculated by summing the abundances of species in which the pathway was reconstructed. Several pathways were enriched in G1 (unfilled, open) or G2 (dashed fill), though these were not statistically significant after multiple test correction.

### DETAILED DESCRIPTION

### I. DEFINITIONS

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The “intestinal tract” or “intestinal” when used as an adjective refers to an individual's stomach, colon, small intestine, large intestine, cecum, and rectum. Synonyms include the gut and the gastrointestinal tract.

“Microbiota” is used to describe the collective population of microorganisms that populate a certain location, such as the gut.

“Microbiome” refers to the collective genomes of a microbiota.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. METHODS FOR DETECTION AND DIAGNOSIS

Methods are provided for detection of liver fibrosis and for detection and/or differential diagnosis of nonalcoholic fatty liver disease. The methods comprise analyzing a biological sample from a subject to determine an intestinal microbiome signature for the subject and inspecting the intestinal microbiome signature relative to a reference intestinal microbiome signature. Studies conducted in support of these methods are now described with reference to Example 1.

In the study detailed in Example 1, a cohort of 86 individuals was selected. In the cohort, 14 individuals had advanced liver fibrosis that was confirmed with liver biopsy. Stool samples from the cohort of individuals were obtained and analyzed via DNA analysis and whole-genome shotgun sequencing. The sequencing data was mapped to microbiome sequence data in a database constructed from genomes of bacteria, archaea, viruses and eukaryotes from NCBI. The relative abundance of bacteria was classified taxonomically into species, genus, family, order, class and phylum to form a training dataset. Also included in the training dataset was sample diversity and sample richness, along with age, gender, race, and body mass index (BMI) of each subject.

The individuals in the study were categorized into two groups based on the severity of fibrosis. The first group (Group 1) of individuals had no fibrosis (Stage 0) or moderate fibrosis (Stages 1 and 2). A second group (Group 2) consisted of those patients whose livers were biopsied to confirm their advanced stages of fibrosis (Stages 3 and 4). Most patients (72) were in Group 1 and 14 patients were part of Group 2. Table 1.1 (below in Example 1) presents demographic, clinical, biochemical and metabolic profile of the entire cohort classified by the advanced fibrosis status. Patients with advanced fibrosis were more likely to be older, Hispanic, diabetic, and had higher ALT, higher AST, lower platelet count, and a higher HbA1 c than those without advanced fibrosis. In addition, although the two groups had similar BMI, patients with advanced fibrosis had higher waist circumference. Table 1.2 (below in Example 1) provides detailed histologic differences in the study cohort classified by the advanced fibrosis status. Patients with advanced fibrosis were more likely to have more severe lobular and portal inflammation and ballooning than those without advanced fibrosis.

Gut microbiome compositions of the patients were determined using whole-genome shotgun sequencing of DNA extracted from their stool samples, as detailed in Example 1. The 86 stool samples yielded an average of 6.58×10⁹ bases per sample (after trimming low-quality bases and removing human sequences). Circulating metabolites and their links to microbial function was also studied, and as described in Example 1, biochemical profiles were generated from serum samples collected from a subset of the cohort (56 patients).

Analysis of the gut microbiome compositions reveals there are differences in the taxonomic composition of stool derived metagenomes between mild/moderate NAFLD versus advanced fibrosis, as shown in Table 1.

TABLE 1
Taxonomic Composition: Relative abundances of top 4 phyla found in
all 86 samples and representative species from the first 3 phyla.

| | G1 Median | G2 Median | p-value |
|---|---|---|---|
| Phylum | | | |
| Firmicutes | 58.81% | 42.61% | 0.01520 |
| Proteobacteria | 1.85% | 4.54% | 0.04004 |
| Bacteroidetes | 23.62% | 28.46% | 0.57840 |
| Actinobacteria | 2.67% | 2.02% | 0.78340 |
| Species | | | |
| Eubacterium rectale | 2.56% | 0.12% | 0.00009* |
| Faecalibacterium prausnitzii | 1.63% | 0.34% | 0.01961 |
| Bacteroides vulgatus | 1.76% | 2.19% | 0.85610 |
| Escherichia coli | 0.29% | 0.99% | 0.44330 |
| Ruminococcus obeum CAG:39 | 0.06% | 0.01% | 0.00005* |
| Ruminococcus obeum | 0.29% | 0.11% | 0.00009* |

*Significant p-value after multiple test correction

As seen in Table 1, at the phylum level, the gut microbiomes in both groups were dominated by members of Firmicutes and Bacteroidetes, followed by Proteobacteria and Actinobacteria in much lower abundances. Furthermore, both Firmicutes and Proteobacteria were differentially abundant across the two groups (p-value <0.05), with Firmicutes being higher in mild/moderate NAFLD (G1) while Proteobacteria was higher in advanced fibrosis (G2). At the species level, Eubacterium rectale (2.5% median relative abundance) and Bacteroides vulgatus (1.7%) were the most abundant organisms in mild/moderate NAFLD (G1) while B. vulgatus (2.2%) and Escherichia coli (1%) were the most abundant in advanced fibrosis (G2). Ruminococcus obeum CAG:39, R. obeum, and E. rectale were significantly lower in advanced fibrosis than mild/moderate NAFLD.

A model utilizing the stool-derived metagenome profiles for the detection of advanced fibrosis was developed. To build a model capable of distinguishing samples belonging to mild/moderate NAFLD and advanced fibrosis, a custom machine learning process that employed Random Forests (RFs) was used. The set of input features for model building consisted of metagenome features and patient metadata features. Features from metagenome data consisted of the number (richness) and relative abundances of 152 constituent species, and microbiome diversity (Shannon diversity). The patient metadata consisted of age, gender, race, and BMI. The first step in building a RF model consisted of training 300 RFs and then selecting the top features from the top-performing model. A feature elimination step was done to optimize the performance of a trained RF, which selected 37 species together with Shannon diversity, Age, and BMI as the most important features. Age was observed to be the top predictor in nearly all of the RFs in the training phase. The statistical significance of the selected features was assessed by Monte Carlo simulation using 10,000 models that were each trained on 40 randomly selected features (p-value <0.006).

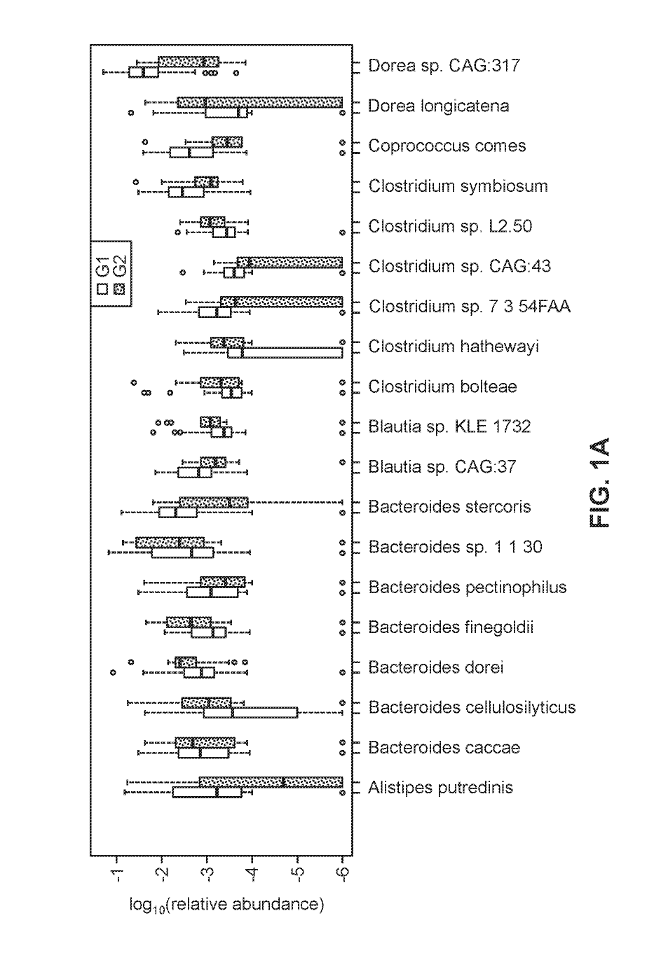

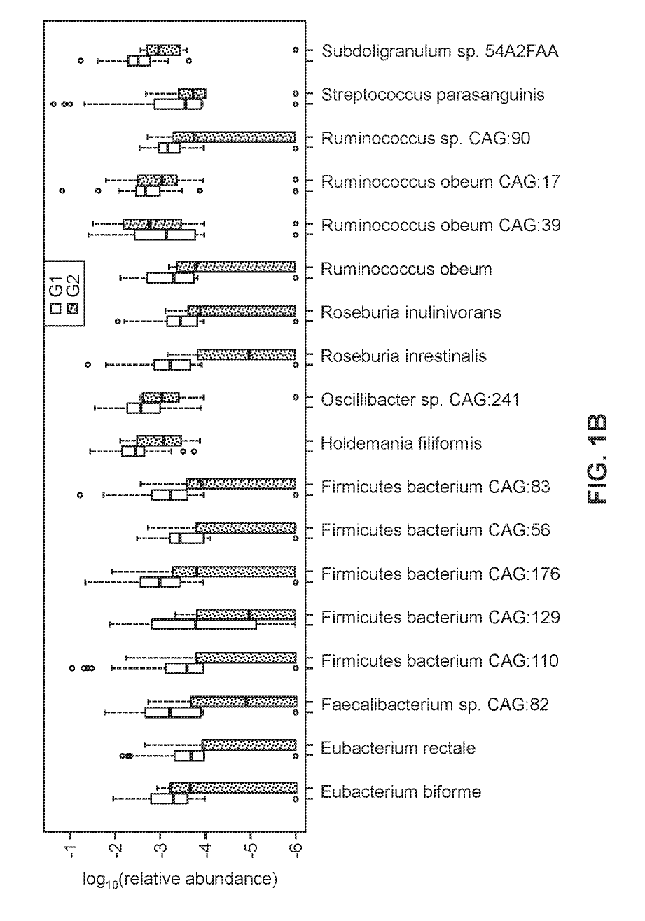

The forty selected features were used to train 50 RFs and the best performing model was selected as the final model. This model had a robust and statistically significant diagnostic accuracy of AUC 0.936. FIGS. 1A-1B are boxplots of the relative abundances of 37 species selected for the random forest model used to distinguish samples in the mild/moderate group (G1) from those in the advanced fibrosis group (G2). Sample diversity and patient age and BMI were also selected as features by the random forest model (boxplots not shown). The range for the AUC estimate (derived from the 50 RFs) was between 0.779 and 0.936.

Table 2 summarizes the 37 species identified using the RF model. Table 2 also provides the mean decrease in Gini index and log ratios of median species abundances in G2 and G1. From the 37 species selected by the optimized model, eight species were more than two-fold more abundant in advanced fibrosis (G2) compared to mild/moderate NAFLD (G1), while 22 species were more than two-fold abundant in mild/moderate NAFLD (G1) compared to advanced fibrosis (G2). Specifically, the median species abundance of 8 species (Dorea sp. CAG: 317, Bacteroides cellulosilyticus, Bacteroides finegoldii, Bacteroides dorei, Streptococcus parasanguinis, Clostridium symbiosum, Clostridium sp. 7 3 54FAA, Clostridium bolteae) were between 2 and 4 fold more abundant in Group 2 than in Group 1 samples. These 8 species are collectively referred to as Group A. The 22 species that were more than two-fold abundant in mild/moderate NAFLD (G1) compared to advanced fibrosis (G2) are collectively referred to herein as Group B. Table 2 also summaries 6 species identified by curation of a genomic database, which are indicated by the Group C species in Table 2.

TABLE 2
Species selected by Random Forest and Database Curation.

| Id. No. and Group | Species | MeanDecreaseGini | log₂(G2/G1) |
|---|---|---|---|
| 1 Group A | Dorea sp. CAG:317 | 0.06 | 2.50 |
| 2 Group A | Bacteroides cellulosilyticus | 0.11 | 1.86 |
| 3 Group A | Bacteroides finegoldii | 0.31 | 1.77 |
| 4 Group A | Bacteroides dorei | 0.18 | 1.59 |
| 5 Group A | Streptococcus parasanguinis | 0.14 | 1.49 |
| 6 Group A | Clostridium symbiosum | 0.15 | 1.35 |
| 7 Group A | Clostridium sp. 7_3_54FAA | 0.16 | 1.34 |
| 8 Group A | Clostridium bolteae | 0.36 | 1.03 |
| | Clostridium hathewayi | 0.14 | 0.88 |
| | Bacteroides stercoris | 0.12 | 0.87 |
| | Bacteroides caccae | 0.10 | 0.68 |
| | Eubacterium biforme | 0.06 | −0.50 |
| 1 Group B | Subdoligranulum sp. 4_3_54A2FAA | 0.05 | −1.00 |
| 2 Group B | Bacteroides sp. 1_1_30 | 0.09 | −1.05 |
| 3 Group B | Faecalibacterium sp. CAG:82 | 0.10 | −1.16 |
| 4 Group B | Clostridium sp. L2-50 | 0.07 | −1.16 |
| 5 Group B | Blautia sp. KLE 1732 | 0.12 | −1.22 |
| 6 Group B | Clostridium sp. CAG:43 | 0.14 | −1.38 |
| 7 Group B | Firmicutes bacterium CAG:56 | 0.14 | −1.39 |
| 8 Group B | Ruminococcus sp. CAG:17 | 0.15 | −1.46 |
| 9 Group B | Ruminococcus obeum | 0.56 | −1.47 |
| 10 Group B | Alistipes putredinis | 0.09 | −1.48 |
| 11 Group B | Roseburia inulinivorans | 0.22 | −1.53 |
| 12 Group B | Ruminococcus sp. CAG:90 | 0.10 | −1.64 |
| 13 Group B | Bacteroides pectinophilus | 0.35 | −1.89 |
| 14 Group B | Roseburia intestinalis | 0.19 | −2.05 |
| 15 Group B | Coprococcus comes | 0.18 | −2.10 |
| 16 Group B | Oscillibacter sp. CAG:241 | 0.36 | −2.26 |
| 17 Group B | Firmicutes bacterium CAG:83 | 0.27 | −2.69 |
| 18 Group B | Dorea longicatena | 0.24 | −2.77 |
| 19 Group B | Firmicutes bacterium CAG:129 | 0.25 | −3.00 |
| 20 Group B | Ruminococcus obeum CAG:39 | 2.37 | −3.53 |
| 21 Group B | Blautia sp. CAG:37 | 0.11 | −3.82 |
| 22 Group B | Eubacterium rectale | 0.68 | −4.40 |
| | Firmicutes bacterium CAG:176 | 0.05 | ND* |
| | Firmicutes bacterium CAG:110 | 0.13 | ND |
| | Holdemania filiformis | 0.21 | ND |
| 1 Group C | Agathobacter rectalis | 0.63 | −2.40 |
| 2 Group C | Blautia sp. KLE 1732 | 0.18 | −1.85 |
| 3 Group C | Roseburia inulinivorans | 0.19 | −2.07 |
| 4 Group C | Oscillibacter (genus) | 0.16 | −1.54 |
| 5 Group C | Eubacterium ramulus | 0.30 | −1.63 |
| 6 Group C | Blautia sp. GD8 | 0.18 | −1.77 |

*The log ratio was not determined (ND) for a few species due to zero median values in G2.

Accordingly, a method for the differential diagnosis of the type of non-alcoholic fatty liver disease (NAFLD) is provided. The method comprises determining an intestinal microbiome signature of the subject, wherein a diagnosis of stage 3-4 fibrosis is indicated by one or more of the following criterion:
(a) the intestinal microbiome signature of the subject having a relative abundance of a bacterial species that is at least two-fold higher than the relative abundance of the bacterial species in a reference intestinal microbiome signature, wherein the bacterial species is selected from the group consisting of Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae (Group A); or
(b) the intestinal microbiome signature of the subject having a relative abundance of a bacterial species that is at least two-fold lower than the relative abundance of the bacterial species in a reference intestinal microbiome signature, wherein the bacterial species is selected from the group consisting of Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alistipes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG:83, Dorea longicatena, Firmicutes bacterium CAG: 129, Ruminococcus obeum CAG:39, Blautia sp. CAG:37, Eubacterium rectale, Firmicutes bacterium CAG:176, Firmicutes bacterium CAG:110, and Holdemania filiformis (Group B); or
(c) the intestinal microbiome signature of the subject having a relative abundance of a bacterial species that is at least about two-fold lower or at least about 2.5-fold lower than the relative abundance of the bacterial species in a reference intestinal microbiome signature, wherein the bacterial species is selected from the group consisting of Agathobacter rectalis (Eubacterium rectale), Blautia sp. KLE 1732, Roseburia inulinivorans, Oscillibacter (genus), Eubacterium ramulus, and Blautia sp. GD8 (Group C); and/or
(d) the intestinal microbiome signature of the subject having (i) a relative abundance of at least two bacterial species in Table 2 that is at least two-fold higher than the relative abundance of the same two bacterial species in a reference intestinal microbiome signature, (ii) a relative abundance of at least two bacterial species in Table 2 that is at least two-fold lower than the relative abundance of the same two bacterial species in a reference intestinal microbiome signature; and the sum of the mean decrease in Gini index of the at least two bacterial species in (i) and of the at least two bacterial species in (ii) is greater than and/or greater than or equal to 0.5.

Diagnosis of stage 3-4 fibrosis is indicated by both (a) and (b) in some embodiments, or alternatively, the diagnosis of stage 3-4 fibrosis is indicated by at least two of the bacterial species of Group A having a relative species abundance in the subject microbiome signature that is at least two-fold higher than in the reference microbiome signature.

The diagnosis of stage 3-4 fibrosis may also be indicated by at least two of the bacterial species of Group B having a relative species abundance in the subject microbiome signature that is at least two-fold lower than in the reference microbiome signature.

The diagnosis of stage 3-4 fibrosis may also be indicated by at least one or at least two of the bacterial species of Group C having a relative species abundance in the subject microbiome signature that is at least two-fold lower or at least about 2.5-fold lower than in the reference microbiome signature.

Diagnosis of stage 3-4 fibrosis, in other embodiments, is indicated by both (a) and (c), by both (a) and (c), by both (a) and (d), by both (b) and (c), or by both (b) and (d), by three of (a), (b), (c) and (d), or by all of (a), (b), (c), and (d).

The diagnosis of stage 3-4 fibrosis may also be indicated by the intestinal microbiome signature of the subject having a relative abundance of a bacterial species that is at least two-fold lower than the relative abundance of the bacterial species in a reference intestinal microbiome signature, wherein the bacterial species is selected from the group consisting of Oscillibacter_sp._CAG.241, Firmicutes bacterium_CAG.129, Firmicutes bacterium_CAG.170, Ruminococcus_obeum, Bacteroides_pectinophilus, Holdemania_jiliformis, and Firmicutes_bacterium_CAG.83.

The diagnosis of stage 3-4 fibrosis may also be indicated by the intestinal microbiome signature of the subject having a relative abundance of a bacterial species that is at least about 2-fold lower or at least about 2.5-fold than the relative abundance of the bacterial species in a reference intestinal microbiome signature, wherein the bacterial species is selected from the group consisting of Agathobacter rectalis (Eubacterium rectale), Blautia sp. KLE 1732, Roseburia inulinivorans, Oscillibacter (genus), Eubacterium ramulus, and Blautia sp. GD8 (Group C).

In another embodiment, a method for the differential diagnosis of the type of non-alcoholic fatty liver disease (NAFLD) comprises determining an intestinal microbiome signature of the subject as described herein, wherein each bacterial species in the intestinal microbiome signature has a mean decrease in Gini index, and wherein the sum of the mean decrease in Gini index for at least n of the bacterial species is greater than or equal to 0.5 is indicative of NASH.

In another embodiment, a method for the diagnosis of stage 3-4 fibrosis comprises determining an intestinal microbiome signature of the subject as described herein, wherein each bacterial species in the intestinal microbiome signature has a mean decrease in Gini index, and wherein the sum of the mean decrease in Gini index for at least n of the bacterial species is greater than or equal to 0.5 is indicative of advanced (stage 3-4) liver fibrosis.

In another embodiment, substantially non-invasive method for assessing risk of progression to liver cirrhosis in a subject having an intestinal microbiome signature and diagnosed with non-alcoholic fatty liver disease (NAFLD) is contemplated and provided. The method comprises, in one embodiment, determining from the intestinal microbiome signature of the subject (i) a relative abundance of at least two bacterial species in Table 2 that is at least two-fold higher than the relative abundance of the same two bacterial species in a reference intestinal microbiome signature and (ii) a relative abundance of at least two bacterial species in Table 2 that is at least two-fold lower than the relative abundance of the same two bacterial species in a reference intestinal microbiome signature. A mean decrease in Gini index for the at least two bacterial species in (i) and (ii) is obtained or determined. The sum of the mean decrease in Gini index for each of the bacterial species in (i) and (ii) is determined, and if the sum of the mean decrease in Gini index is greater than 0.5 risk of progression to liver cirrhosis is indicated.

In embodiments of the methods wherein a summation of mean decrease in Gini index is determined for one or more species in an intestinal microbiome signature, the summation value can be greater than 0.25, 0.3, 0.4, 0.5, 0.6, 0.7, 0.8, 0.9 or 1.0 or can be greater than or equal to 0.25, 0.3, 0.4, 0.5, 0.6, 0.7, 0.8, 0.9 or 1.0. Further, in embodiments of the methods wherein a summation of mean decrease in Gini index is determined for one or more species in an intestinal microbiome signature, the one or more species, or the value of n can be any of the ranges or values of n set forth herein, an in certain embodiments is 2, 3, 4, 5, 6, 7, 8, 9, or 10.

In order to further validate the existence of a signature to distinguish between groups mild/moderate NAFLD (G1) and advanced fibrosis (G2), an orthogonal machine learning method based on linear Support Vector Machine (SVM) was used to build a classifier from the same input feature set; this resulted in a model whose final feature set had a high degree of concordance with the features in the RF based model and with a similarly high AUC. Example 1 describes the model in detail. The trained SVM selected 18 species as the predictors, which are shown in Table 3. Twelve of the species overlapped with the species selected by the Random Forest method, and these overlapping species are identified in bold in Table 3.

TABLE 3
Top features selected by Linear SVM

| Rank | Feature | coef |
|---|---|---|
| 1 | [symbol] | 1.02 |
| 2 | [symbol] bacterium CAG:129 | −0.90 |
| 3 | [symbol] bacterium CAG:24 | −0.89 |
| 4 | Age | 0.83 |
| 5 | Asian | −0.80 |
| 6 | [symbol] sp. CAG:43 | −0.80 |
| 7 | [symbol] sp. CAG:241 | −0.80 |
| 8 | Firmicutes bacterium CAG:103 | 0.77 |
| 9 | [symbol] sp. CAG:90 | −0.77 |
| 10 | [symbol] CAG:39 | −0.76 |
| 11 | [symbol] | −0.72 |
| 12 | Alistipes shahii | 0.72 |
| 13 | Clostridium symbiosum | 0.66 |
| 14 | [symbol] sp._{—}7_{—}3_{—}54FAA | 0.61 |
| 15 | Clostridium sp._CAG:58 | −0.58 |
| 16 | [symbol] sp._{—}1_{—}1_{—}30 | −0.56 |
| 17 | Bacteroides sp._1_1_14 | −0.43 |
| 18 | Female | 0.42 |
| 19 | Escherichia coli | −0.40 |
| 20 | [symbol] | 0.38 |
| 21 | [symbol] sp._{—}CAG:37 | −0.35 |
| 22 | Hispanic | 0.14 |

*The species identified by bold font overlap with those identified using the Random Forest Model.

Twelve of the species identified by linear SVM (Table 3) overlapped with the species selected by the Random Forest method (Table 2), and the 12 common species are listed in Table 4 below. Of the 12 species listed in Table 4, 4 species (Bacteroides finegoldii, Clostridium sp. 7_3_54FAA, Clostridium symbiosum, and Streptococcus parasanguinis) were observed to be more abundant in Group 2 samples based on the log fold change in their median abundances between the two groups (last column of Table 2). There are roughly 20 species that are between 2 and 16 fold more abundant in Group 1 than in Group 2.

TABLE 4

| Species | |
|---|---|
| No. | Species |
| 1 | Bacteroides finegoldii |
| 2 | Bacteroides sp. 1_1_30 |
| 3 | Blautia sp. CAG:37 |
| 4 | Clostridium sp. 7_3_54FAA |
| 5 | Clostridium sp. CAG:43 |
| 6 | Clostridium symbiosum |
| 7 | Eubacterium rectale |
| 8 | Firmicutes bacterium CAG:129 |
| 9 | Oscillibacter sp. CAG:241 |
| 10 | Ruminococcus obeum CAG:39 |
| 11 | Ruminococcus sp. CAG:90 |
| 12 | Streptococcus parasanguinis |

Database curation identified species identified in Table 5 that distinguish advanced fibrosis from no fibrosis or moderate fibrosis. As seen in Table 5, the identified species are at least about two fold, or at least about 2.5 fold lower in patients with advanced fibrosis relative to patients with no or moderate fibrosis. These species in Table 5 belong to order Clostridiales in Firmicutes phylum of bacteria.

TABLE 5
Bacterial Species to identify Advanced Fibrosis

| | Bacteria relative abundance | | | | |
|---|---|---|---|---|---|
| | No/moderate | Advanced | | False | Adjusted |
| Species/genus | fibrosis | fibrosis | FC change | discovery rate | p value |
| Agathobacter rectalis * | 0.0619 | 0.0117 | 5.3 | 0.0002 | 0.004 |
| Blautia sp. KLE 1732 | 0.0042 | 0.0011 | 3.6 | 0.0009 | 0.026 |
| Roseburia inulinivorans | 0.0081 | 0.0019 | 4.2 | 0.0009 | 0.039 |
| Oscillibacter (genus) | 0.0073 | 0.0025 | 2.9 | 0.0022 | 0.058 |
| Eubacterium ramulus | 0.0028 | 0.0009 | 3.1 | 0.0018 | 0.073 |
| Blautia sp. GD8 | 0.0044 | 0.0013 | 3.4 | 0.0021 | 0.092 |

* Agathobacter rectalis is also named as Eubacterium rectale.

Two sources of data were used to validate the performance of the metagenome derived models to differentiate advanced fibrosis from no advanced fibrosis. (i) Age is a major effect modifier of both microbiome as well as advanced fibrosis. In order to examine that the metagenome-derived model was not biased by age, the RF model was applied to a previously published and well-phenotyped twin cohort dataset (Loomba, R. et al. Gastroenterology, 149(7):1784-1793 (2015)). A priori, a single twin (as twins are known to have a significantly shared microbiome) was selected from a pair of twins who were 60 years of age or older and healthy based upon a normal liver fat content without hepatic steatosis as determined by MRI PDFF <5% (no NAFLD) and absence of fibrosis as determined by an MRE <3 Kpa (no fibrosis). The AUC of the predictions made by the trained RF on data from these uniquely well-characterized 28 healthy older twin individuals remained consistent and robust with an AUC of 0.89 (p-value <0.0001, Monte Carlo simulation using permuted class labels).

Further validation of the framework in an independent group of patients was conducted by establishing an age-balanced group of patients with NAFLD cirrhosis and those without fibrosis in which a nearly equal number of patients that were all over the age of 60 with either NAFLD cirrhosis (N=14) or no fibrosis (N=16) were studied. Patient data and species abundances from this NASH cirrhosis and control samples were used to train another RF model that had an AUC of 0.80. From the nine microbial species selected by this model shown in Table 6 seven overlap (p-value <0.0008) with the 37 species selected by the original RF model. Table 6 also shows the mean decrease in Gini index and the log ratios of median species abundances in G2 and G1.

TABLE 6
Species selected by a Random Forest model trained
with data from an age-balanced group of patients.

| | Mean Decrease | |
|---|---|---|
| Species | Gini | Log2(G2/G1) |
| Oscillibacter_{—}sp._{—}CAG.241* | 2.41 | −5.11 |
| Firmicutes_{—}bacterium_{—}CAG.129 | 1.25 | −4.57 |
| Firmicutes_{—}bacterium_{—}CAG.170 | 1 | −3.73 |
| [Ruminococcus]_{—}obeum | 1.62 | −3.68 |
| [Bacteroides]_{—}pectinophilus | 0.36 | −3.02 |
| Oscillibacter_sp._1.3 | 0.94 | −2.78 |
| Holdemania_{—}filiformis | 1.21 | −2.47 |
| Firmicutes_{—}bacterium_{—}CAG.83 | 1.47 | −2.33 |
| Firmicutes_bacterium_CAG.103 | 0.79 | −1.44 |

*Species in bold overlap with the species selected in the RF model (Table 2).

Accordingly, a method for diagnosing advanced fibrosis due to NAFLD by determining a microbiome signature in a patient sample, using for example a diagnostic test for detection of a panel of microbiome-derived biomarkers, is contemplated. The method provides for non-invasive detection of advanced fibrosis and for the screening of advanced fibrosis or cirrhosis, using a sample from the gastrointestinal tract, such as a stool sample. The gut microbiomes in NAFLD is dominated by members of Firmicutes and Bacteroidetes, followed by Proteobacteria and Actinobacteria in much lower abundances (Table 1 above). As the disease progresses from mild/moderate NAFLD to advanced fibrosis, the Proteobacteria take over from the Firmicutes suggesting that Firmicutes may play a role in transition to advanced fibrosis and once advanced fibrosis sets in the Proteobacteria take over.

At the species level, E. rectale (2.5% median relative abundance) and B. vulgatus (1.7%) were the abundant organisms in mild/moderate NAFLD while B. vulgatus (2.2%) and E. coli (1%) were the abundant in advanced fibrosis. None of the patients in with advanced fibrosis had ascites or any evidence of hepatic decompensation but still had high E. coli abundance. This increased abundance of E. coli in advanced fibrosis has potential clinical implications. These data suggest that E. coli dominance occurs much earlier in the stage of fibrosis progression and supports the hypothesis that dysbiosis may precede development of portal hypertension.

Accordingly, in one embodiment, methods to detect liver fibrosis and for the differential diagnosis of the type of non-alcoholic fatty liver disease (NAFLD) in a subject comprise analyzing a biological sample from the subject to determine an intestinal microbiome signature for the subject, wherein a microbiome signature comprised of E. coli and at least n bacterial species identified in Table 2, Table 3, Table 4 or Table 5 indicates liver fibrosis or nonalcoholic steatohepatitis.

The data described herein establishes the use of metagenomics sequencing rather than 16S rDNA gene sequencing as an approach to detection of liver fibrosis. The data reveals, from the analysis of stool metagenomes from a well phenotyped NAFLD cohort, 37 microbial species that are differentially present in the different stages of the disease. Microbial biomarkers can be used to diagnose metabolic and fibrotic diseases and provide a tool to determine stage of liver disease. The metagenomics signature may also be used in conjunction with other non-invasive serum/plasma or imaging based tests to detect fibrosis, advanced fibrosis and cirrhosis.

Accordingly, a method to detect liver fibrosis in a subject is provided. The method comprises analyzing a biological sample from a subject to determine an intestinal microbiome signature for the subject; and inspecting the intestinal microbiome signature relative to a reference intestinal microbiome signature to detect presence or absence of liver fibrosis.

Also provided are methods for the differential diagnosis of the type of non-alcoholic fatty liver disease (NAFLD) in a subject. In one method, it is determined whether at least n bacterial species identified in Table 2, Table 3, Table 4 or Table 5 is present in intestinal microflora of the subject, wherein presence of the at least n bacterial species identified in Table 2, Table 3, Table 4 or Table 5 in the intestinal microflora indicates nonalcoholic steatohepatitis (NASH). In another method, intestinal microflora of the subject is analyzed to determine an intestinal microbiome signature for the subject; and the intestinal microbiome signature is inspected to determine whether at least n bacterial species identified in Table 2, Table 3, Table 4 or Table 5 is present in the signature. Presence of at least n bacterial species identified in Table 2, Table 3, Table 4 or Table 5 in the intestinal microbiome signature indicates nonalcoholic steatohepatitis (NASH).

A method for the differential diagnosis of the type of non-alcoholic fatty liver disease (NAFLD) in a subject, comprising determining whether at least n bacterial species identified in Table 2, Table 3, Table 4 and/or Table 5 is present in intestinal microflora of the subject, wherein presence of the at least n bacterial species identified in Table 2, Table 3, Table 4 and/or Table 5 in the intestinal microflora indicates nonalcoholic steatohepatitis (NASH).

In one embodiment, n is at least 8 and is comprised of the bacterial species in Group A (Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae. In some cases, the intestinal microbiome signature comprised of the Group A bacterial species have a relative abundance of each bacterial species in the signature at least two-fold higher than a relative abundance of the Group A bacterial species in a reference intestinal microbiome signature.

In another embodiment, n is at least 9 and additionally comprises one or more of the bacterial species in Group B (Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alistipes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG:83, Dorea longicatena, Firmicutes bacterium CAG:129, Ruminococcus obeum CAG:39, Blautia sp. CAG:37, Eubacterium rectale, Firmicutes bacterium CAG:176, Firmicutes bacterium CAG:110, and Holdemania fihformis). In some cases, the Group B bacterial species in the intestinal microbiome signature have a relative abundance at least two-fold lower than a relative abundance of the Group B bacterial species in a reference intestinal microbiome signature.

In another embodiment, n is at least 2 and comprises one or more of the bacterial species in Group C (Agathobacter rectalis (Eubacterium rectale), Blautia sp. KLE 1732, Roseburia inulinivorans, Oscillibacter (genus), Eubacterium ramulus, and Blautia sp. GD8).

The method contemplated herein for the differential diagnosis of the type of non-alcoholic fatty liver disease (NAFLD) in another embodiment comprises determining an intestinal microbiome signature of the subject, wherein a diagnosis of stage 3-4 fibrosis is indicated by one or more of the following criterion:
(a) the intestinal microbiome signature of the subject having a relative abundance of a bacterial species that is at least two-fold higher than the relative abundance of the bacterial species in a reference intestinal microbiome signature, wherein the bacterial species is selected from the group consisting of Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae (Group A); or
(b) the intestinal microbiome signature of the subject having a relative abundance of a bacterial species that is at least two-fold lower than the relative abundance of the bacterial species in a reference intestinal microbiome signature, wherein the bacterial species is selected from the group consisting of Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alistipes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG:83, Dorea longicatena, Firmicutes bacterium CAG: 129, Ruminococcus obeum CAG:39, Blautia sp. CAG: 37, Eubacterium rectale, Firmicutes bacterium CAG:176, Firmicutes bacterium CAG:110, and Holdemania filiformis (Group B); or
(c) the intestinal microbiome signature of the subject having a relative abundance of a bacterial species that is at least about two-fold lower or at least about 2.5-fold lower than the relative abundance of the bacterial species in a reference intestinal microbiome signature, wherein the bacterial species is selected from the group consisting of Agathobacter rectalis (Eubacterium rectale), Blautia sp. KLE 1732, Roseburia inulinivorans, Oscillibacter (genus), Eubacterium ramulus, and Blautia sp. GD8 (Group C); and/or
(d) the intestinal microbiome signature of the subject having (i) a relative abundance of at least two bacterial species in Table 2 that is at least two-fold higher than the relative abundance of the same two bacterial species in a reference intestinal microbiome signature, (ii) a relative abundance of at least two bacterial species in Table 2 that is at least two-fold lower than the relative abundance of the same two bacterial species in a reference intestinal microbiome signature; and the sum of the mean decrease in Gini index of the at least two bacterial species in (i) and of the at least two bacterial species in (ii) is greater than and/or greater than or equal to 0.5.

Diagnosis of stage 3-4 fibrosis is indicated by both (a) and (b) in some embodiments, or alternatively, the diagnosis of stage 3-4 fibrosis is indicated by at least two of the bacterial species listed in (a) having a relative species abundance in the subject microbiome signature that is at least two-fold higher than in the reference microbiome signature.

In yet another embodiment, the diagnosis of stage 3-4 fibrosis is indicated by at least two of the bacterial species listed in (b) having a relative species abundance in the subject microbiome signature that is at least two-fold lower than in the reference microbiome signature, and/or one or more of the bacterial species listed in (c) having a relative species abundance in the subject microbiome signature that is at least 2-fold lower or 2.5-fold than in the reference microbiome signature.

In still another embodiment, the diagnosis of stage 3-4 fibrosis is indicated by the intestinal microbiome signature of the subject having a relative abundance of a bacterial species that is at least two-fold lower than the relative abundance of the bacterial species in a reference intestinal microbiome signature, wherein the bacterial species is selected from the group consisting of Oscillibacter_sp._CAG.241, Firmicutes_bacterium_CAG.129, Firmicutes_bacterium_CAG.170, Ruminococcus_obeum, Bacteroides_pectinophilus, Holdemania_filiformis, and Firmicutes_bacterium_CAG. 83.

In another aspect, a method for the differential diagnosis of the type of non-alcoholic fatty liver disease (NAFLD) in a subject, comprising analyzing intestinal microflora of the subject to determine an intestinal microbiome signature for the subject; and inspecting the intestinal microbiome signature to determine whether at least n bacterial species identified in Table 2, Table 3, Table 4 and/or Table 5 is present in the signature. Presence of the at least n bacterial species identified in Table 2, Table 3, Table 4 and/or Table 5 in the intestinal microbiome signature indicates nonalcoholic steatohepatitis (NASH).

In another aspect, a substantially non-invasive method for assessing risk of progression to liver cirrhosis in a subject having an intestinal microbiome signature and diagnosed with non-alcoholic fatty liver disease (NAFLD), comprising determining whether at least n bacterial species identified in Table 2, Table 3, Table 4 and/or Table 5 is present in the intestinal microbiome signature of the subject, wherein presence of the at least n bacterial species identified in Table 2, Table 3, Table 4 and/or Table 5 in the intestinal microbiome signature indicates risk of progression to liver cirrhosis.

In still another aspect, an assay method to differentiate nonalcoholic fatty liver (NAFL) from nonalcoholic steatohepatitis (NASH) in a subject with non-alcoholic fatty liver disease (NAFLD) is provided. The method comprises analyzing intestinal microflora of the subject to determine an intestinal microbiome signature for the subject; and inspecting the intestinal microbiome signature to determine whether at least n bacterial species identified in Table 2 is present in the signature, wherein presence of the at least n bacterial species identified in Table 2 in the intestinal microbiome signature indicates nonalcoholic steatohepatitis (NASH).

The sample from the subject to be analyzed can be a stool sample, an intestinal mucosal sample or a sample of the intestinal contents. Use of a stool sample offers the advantage of a non-invasive method.

The sample is analyzed to ascertain its microbiome signature by any of a variety of techniques known to skilled artisans, such as those described in Sekirov, I., et al. Physiol. Rev, 90:859-904 (2010). For example, in one embodiment, the biological sample is applied to a test panel that detects bacterial species, and in one embodiment is a test panel for detection of at least n bacterial species identified in Table 2, Table 3, Table 4 and/or Table 5. The test panel can be a culture-based panel, a culture-independent technique such as a molecular-based panel using bacterial 16S ribosomal RNA identification, full-length 16S rRNA sequencing panel (via Sanger sequencing), a DNA microarray panel, etc. Metagenomic sequencing using, for example, next-generation sequencing techniques such as sequencing by synthesis (SBS) chemistry, ion semiconductor (ion torrent) sequencing, pyrosequencing, sequencing by ligation, and other methods known to those of skill in the art, can also be used to determine the microbiome signature. Large-scale shotgun-type metagenomic sequencing may be employed, and targeted metagenomic sequencing is also suitable. In one embodiment, the bacterial species in the microbiome signature are identified using pyrosequencing, such as multitag pyrosequencing. Other analysis methods include denaturing gel electrophoresis, terminal restriction fragment length polymorphisms, ribosomal intergenic spacer analysis, FISH and qPCR. FISH and qPCR use fluorescently labeled oligonucleotide probes that hybridize to 16S rRNA sequences unique to the targeted bacterial species. The analytical techniques can be used in combination, for example, FISH and qPCR. Other identification techniques include microscopy, metabolite identification, Gram staining, flow cytometry, and immunological assays.

The microbiome signature that is determined from analysis of the sample can be based on absolute amounts of each species identified or on relative abundance of species identified. In one embodiment a relative abundance of bacterial species in the intestinal microbiome signature is used to define the signature, where a median abundance of each bacterial species in sample relative to a median abundance of each bacterial species in a reference intestinal microbiome signature is used.

In another embodiment, analyzing further comprises defining an intestinal microbiome signature according to the presence or absence of the at least n bacterial species identified in Table 2, Table 3, Table 4 and/or Table 5.

After determining the microbiome signature, it is analyzed relative to, inspected relative to, or compared to a reference intestinal microbiome signature. The reference intestinal microbiome signature can vary according to the disorder to be diagnosed, and in one embodiment, the reference intestinal microbiome signature is obtained from a population of subjects without liver fibrosis. In another embodiment, the reference intestinal microbiome signature is obtained from obtained from a population of subjects with liver fibrosis. In one embodiment, the population of subjects has advanced liver fibrosis, and in other embodiments, the population of subjects have nonalcoholic fatty liver (NAFL) or nonalcoholic steatohepatitis (NASH). Comparison of the obtained intestinal microbiome signature to a reference signature permits diagnosis of, for example, a stage of liver fibrosis, such as advanced liver fibrosis. It will be appreciated that liver fibrosis is not an independent disease but is a histological change present in a number diseases. For example, chronic viral hepatitis B and C are common causes of liver fibrosis. Severity of liver fibrosis is typically classified into five stages, designated S0, S1, S2, S3, and S4. S0 is no fibrosis. S4 is cirrhosis. In between, S1 is mild fibrosis at the portal area; S2 is moderate stage of fibrosis between the portal areas without destruction of the lobular structure. S3 is severe fibrosis, observed by fibrostic bridging between portal areas and between portal areas and center veins. At S4, in addition to the observations of S2 there are pseudo-lobules formed.

In other embodiments, based on inspection of the intestinal microbiome signature a differential diagnosis of the type of non-alcoholic fatty liver disease (NAFLD) is determined. The intestinal microbiome signature is determined and inspected to ascertain whether at least n bacterial species identified in Table 2, Table 3, Table 4 or Table 5 is present in the signature. Presence of the at least n bacterial species identified in Table 2, Table 3, Table 4 or Table 5 in the intestinal microbiome signature indicates nonalcoholic steatohepatitis (NASH). Alternatively, absence of the at least n bacterial species identified in Table 2, Table 3, Table 4 or Table 5 in the intestinal microbiome signature indicates nonalcoholic fatty liver (NAFL).

The intestinal microbiome signature determined from the sample may contain n bacterial species identified in Table 2, where n is selected from the group consisting of one (1), two (2), three (3), four (4), five (5), six (6), seven (7), eight (8), nine (9), ten (10), eleven (11), twelve (12), thirteen (13), fourteen (14), fifteen (15), sixteen (16), seventeen (18), nineteen (19) and twenty (20). Alternatively, n is selected from the group consisting of about 1-30, about 1-25, about 5-30, about 5-25, about 10-30, and about 10-25. Alternatively, n is selected from the group consisting of greater than one (1), greater than two (2), greater than three (3), greater than four (4), greater than five (5), greater than six (6), greater than seven (7), greater than eight (8), greater than nine (9), greater than ten (10), greater than eleven (11), greater than twelve (12), greater than thirteen (13), greater than fourteen (14), greater than fifteen (15), greater than sixteen (16), greater than seventeen (18), greater than nineteen (19) and greater than twenty (20). Alternatively, n is selected from the group consisting of at least one (1), at least two (2), at least three (3), at least four (4), at least five (5), at least six (6), at least seven (7), at least eight (8), at least nine (9), at least ten (10), at least eleven (11), at least twelve (12), at least thirteen (13), at least fourteen (14), at least fifteen (15), at least sixteen (16), at least seventeen (18), at least nineteen (19) and at least twenty (20). Alternatively, n corresponds to the species in Group A and/or Group B and/or Group C of Table 2.

The intestinal microbiome signature determined from the sample may contain n bacterial species identified in Table 3, where n is selected from the group consisting of one (1), two (2), three (3), four (4), five (5), six (6), seven (7), eight (8), nine (9), ten (10), eleven (11), twelve (12), thirteen (13), fourteen (14), fifteen (15), sixteen (16), seventeen (18), nineteen (19) and twenty (20). Alternatively, n is selected from the group consisting of about 1-30, about 1-25, about 5-30, about 5-25, about 10-30, and about 10-25. Alternatively, n is selected from the group consisting of greater than one (1), greater than two (2), greater than three (3), greater than four (4), greater than five (5), greater than six (6), greater than seven (7), greater than eight (8), greater than nine (9), greater than ten (10), greater than eleven (11), greater than twelve (12), greater than thirteen (13), greater than fourteen (14), greater than fifteen (15), greater than sixteen (16), greater than seventeen (18), greater than nineteen (19) and greater than twenty (20).

The intestinal microbiome signature determined from the sample may contain n bacterial species identified in Table 4, where n is selected from the group consisting of one (1), two (2), three (3), four (4), five (5), six (6), seven (7), eight (8), nine (9), ten (10), eleven (11), and twelve (12). Alternatively, n is selected from the group consisting of about 1-12, about 1-11, about 1-10, about 1-9, about 1-8, about 1-7, about 1-6, about 1-5, about 1-4, about 1-3, and about 1-2. Alternatively, n is selected from the group consisting 5-12, 5-11, 5-10, and 5-9 of the species in Table 4.

The intestinal microbiome signature determined from the sample may contain n bacterial species identified in Table 5, where n is selected from the group consisting of one (1), two (2), three (3), four (4), five (5), and six (6). Alternatively, n is selected from the group consisting of about 1-6, about 1-5, about 1-4, about 1-3, and about 1-2. Alternatively, n is selected from the group consisting 6 or fewer, 5 or less, 4 or less, 3 or less, 2 or less or just one of the species in Table 5.

In another embodiment, the intestinal microbiome signature determined from the sample comprises (i) at least one of Bacteroides finegoldii and Bacteroides sp. 1_1_30; (ii) Blautia sp. CAG:37; (iii) at least one of Clostridium sp. 7_3_54FAA, Clostridium sp. CAG:43, and Clostridium symbiosum; (iv) Eubacterium rectale; (v) Firmicutes bacterium CAG:129, (vi) Oscillibacter sp. CAG:241, (vii) at least one of Ruminococcus obeum CAG:39 and Ruminococcus sp. CAG:90, and (viii) Streptococcus parasanguinis.

The test panel or technique used to determine the microbiome signature is correspondingly designed for detection of the n bacterial species. Techniques suitable for test panels for detection of the n bacterial species are mentioned above. In one example, a microarray comprising nucleic acid sequences with binding affinity for the n bacterial species set forth in Table 2, Table 3, Table 4 or Table 5 is provided. The nucleic acid is DNA, cDNA, RNA, mRNA, or rRNA. In another example, nucleic acid from the n bacterial species is isolated and amplified via real-time polymerase chain reaction, reverse transcription polymerase chain reaction, isothermal amplification, or the like, for detection of the amplicons. In another example, the test panel identifies presence or absence of n bacterial species using nucleic acid sequencing, including Sanger sequencing or pyrosequencing. The nucleic acid sequencing can be total DNA sequencing or sequencing of the complete 16S rRNA gene or sequencing of a hypervariable region of the 16S rRNA gene, such as the V6 region.

In another embodiment, the intestinal microbiome signature is based on bacterial metabolic products from intestinal microflora or on proteins in intestinal microflora, and a bacterial microbiome signature is determined from the metabolic products or the proteins.

It will be appreciated that the methods described herein provide a substantially non-invasive method for assessing risk of progression to liver cirrhosis in a subject having an intestinal microbiome signature and diagnosed with non-alcoholic fatty liver disease (NAFLD), comprising determining whether at least n bacterial species identified in Table 2, Table 3, Table 4 or Table 5 is present in the intestinal microbiome signature of the subject, wherein presence of the at least n bacterial species identified in Table 2, Table 3, Table 4 or Table 5 in the intestinal microbiome signature indicates risk of progression to liver cirrhosis.

The techniques and methods described also provide an assay method to differentiate nonalcoholic fatty liver (NAFL) from nonalcoholic steatohepatitis (NASH) in a subject with non-alcoholic fatty liver disease (NAFLD). The method comprises analyzing intestinal microflora of the subject to determine an intestinal microbiome signature for the subject; and inspecting the intestinal microbiome signature to determine whether at least n bacterial species identified in Table 2, Table 3, Table 4 or Table 5 is present in the signature, wherein presence of the at least n bacterial species identified in Table 2, Table 3, Table 4 or Table 5 in the intestinal microbiome signature indicates nonalcoholic steatohepatitis (NASH). In one embodiment, absence of the at least n bacterial species identified in Table 2, Table 3, Table 4 or Table 5 in the intestinal microbiome signature indicates nonalcoholic fatty liver (NAFL).

### Metabolome Analysis

An analysis of microbial function using metagenome and metabolome data was also conducted. The plausible function of the metagenome derived gut microbiota profile of advanced fibrosis in NAFLD was explored. Metagenome data were used to assess the functional and metabolic potential of the microbial communities associated with the two groups, via a quantification of the relative abundances of protein families and enzymes in the samples and the relative abundances of the pathways reconstructed from species bins generated from assembled data. These data were integrated with serum metabolite data to evaluate microbial metabolism.

Metabolites detected in serum samples include those that are endogenous or of microbial origin (Guo, L., et al., Proc. Natl. Acad. Sci., 112(35):E4901-E4910 (2015)). To further evaluate those metabolites that may be of microbial origin, the full set of metabolites detected from the 56 serum samples were intersected with the set of metabolites predicted from the microbial pathways reconstructed from the stool metagenome data. This comparison resulted in 89 metabolites and included several known to be produced by both host and microbes. A differential analysis identified 11 metabolites whose abundances (peak intensities) are significantly different between mild/moderate NAFLD (G1) and advanced fibrosis (G2) (Wilcoxon rank sum corrected for FDR and α=0.05), and these 11 metabolites are set forth in Table 1.4 below in Example 1. In this set, two metabolites (associated with nucleoside metabolism) were enriched in mild/moderate NAFLD (G1), while nine metabolites (associated with amino acids and carbon metabolism) were enriched in advanced fibrosis (G2). Though its differential abundance was not statistically significant, the metabolite with the highest fold increase in advanced fibrosis (G2) was 3-phenylpropanoate, a metabolite produced by anaerobic bacteria (Wikoff, W. R., et al., Proc. Natl. Acad. Sci. U.S.A., 106(10):3698-3703 (2009); Moss, C. W., et al., Appl. Microbiol., 19(2):375-8 (1970)).

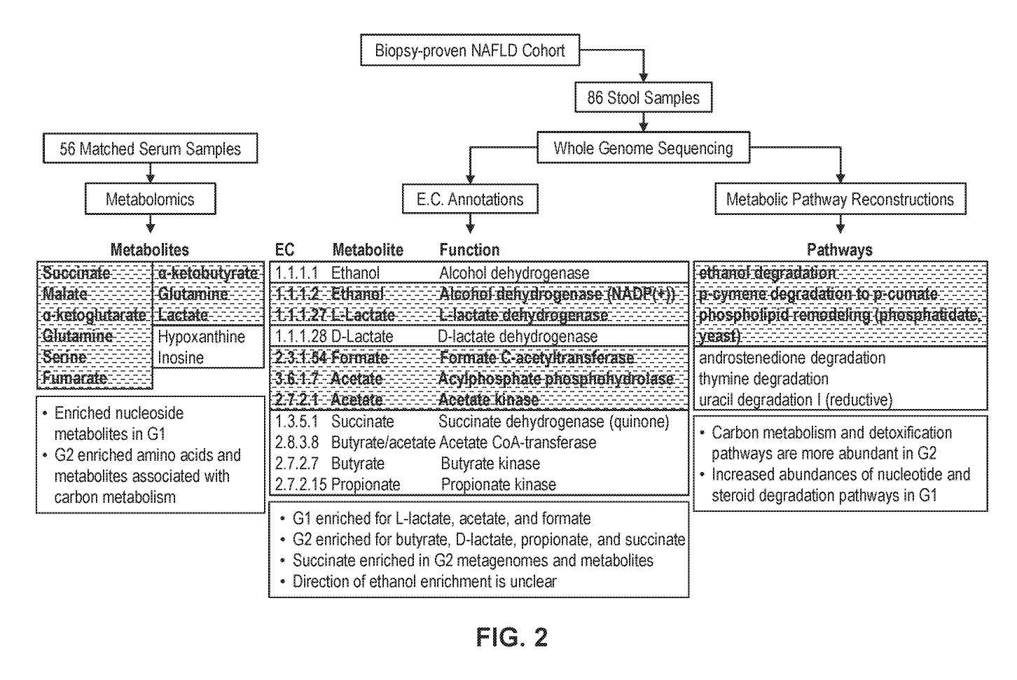

No pathways, protein families, nor enzymes were identified whose differential abundances across mild/moderate NAFLD (G1) and advanced fibrosis (G2) were statistically significant (after multiple test correction). However, an examination of pathway abundances showed that advanced fibrosis (G2) had an increased abundance of pathways associated with carbon metabolism and detoxification, while mild/moderate NAFLD (G1) had an increased abundance of pathways associated with nucleotide and steroid degradation. These findings are illustrated in FIG. 2. An evaluation of the protein families and enzymes associated with Short-Chain Fatty Acid (SCFA) production suggested that mild/moderate NAFLD (G1) had higher abundances of enzymes associated with lactate, acetate, and formate, while advanced fibrosis (G2) had higher abundances of enzymes for butyrate, D-lactate, propionate, and succinate (FIG. 2). The trend for the abundances of ethanol metabolism enzymes in G1 or G2 was not as clear, with enzyme EC 1.1.1.1 (Alcohol dehydrogenase) increased in G2, while enzyme EC 1.1.1.2 (Alcohol dehydrogenase NADP(+)) was increased in G1.

### III. EXAMPLES

The following examples are illustrative in nature and are in no way intended to be limiting.

### Example 1

### Analysis Of Human Gut Microbiota

A cohort of 86 individuals (female 56%) with biopsy-proven NAFLD were classified into two groups: Group 1 with 72 patients with stage 0-2 fibrosis were classified as mild/moderate NAFLD and Group 2 with 14 patients with stage 3-4 fibrosis classified as advanced NAFLD. Table 1.1 provides a summary of the individuals in Group 1 and Group 2.

TABLE 1.1
Baseline Characteristics of patients with biopsy-proven NAFLD

| | | Group 1: | | Group 2: | | |
|---|---|---|---|---|---|---|
| | | Stage 0-2 | | Stage 3-4 | | |
| | | Mild, Moderate | | Advanced | | p-value |
| | All patients | Fibrosis | | Fibrosis | | (Student's |
| Characteristics | N = 86 | N = 72 | | N = 14 | | t-test) |
| Demographics | | | | | | |
| Age (mean ± SD) | 48 ± 1.4 | 49.3 ± 12.6 | | 63.4 ± 3 | | 1.5e−12 |
| Male n (%) | 38 (44.2%) | 36 | (50%) | 2 | (14.3%) | 0.030 |
| White n (%) | 40 (46.5%) | 33 | (40.2%) | 7 | (50%) | 1.000 |
| Hispanic n (%) | 29 (33.7%) | 23 | (31.9%) | 6 | (42.9%) | 0.630 |
| Clinical | | | | | | |
| Type 2 diabetes n (%) | 20 (23.3%) | 14 | (19.4%) | 6 | (42.9%) | 0.126 |
| Anthropometric (mean ± SD) | | | | | | |
| Body mass index (kg/m2) | 31.2 ± 5.5 | 31.0 ± 5.4 | | 32.2 ± 6.0 | | 0.503 |
| Waist circumference (cm) | 102.4 ± 16.3 | 101.5 ± 19.2 | | 107.1 ± 17.3 | | 0.823 |
| Hepatology panel (mean ± SD) | | | | | | |
| AST (U/L) | 41.0 ± 30.0 | 35 ± 24.5 | | 72 ± 36.8 | | 0.002 |
| ALT (U/L) | 57.0 ± 55.2 | 53.8 ± 54.3 | | 73.8 ± 55.2 | | 0.253 |
| AST/ALT | 0.72 | 0.65 | | 0.98 | | |
| Bilirubin, direct (mg/dL) | 0.16 ± 0.12 | 0.13 ± 0.06 | | 0.29 ± 0.23 | | 0.033 |
| Hematology and other laboratory | | | | | | |
| studies (mean ± SD) | | | | | | |

| | | | | |
|---|---|---|---|---|
| White blood cells (1000/mm3) | 6.3 ± 1.7 | 6.3 ± 1.6 | 6.2 ± 2.2 | 0.843 |
| Platelet count (1000/mm3) | 250.5 ± 79.6 | 254.5 ± 64.3 | 230.2 ± 135.2 | 0.521 |
| Total cholesterol (mg/dL) | 190.5 ± 42.3 | 193.9 ± 42.2 | 173.0 ± 39.6 | 0.089 |
| HDL cholesterol (mg/dL) | 48.9 ± 16.0 | 48.9 ± 15.9 | 48.6 ± 17.1 | 0.942 |
| LDL cholesterol (mg/dL) | 112.4 ± 36.2 | 116 ± 34.7 | 94.9 ± 39.8 | 0.178 |
| Triglycerides (mg/dL) | 159.9 ± 95.8 | 160.6 ± 98.3 | 156.6 ± 84.1 | 0.565 |
| HbA1c (%) | 6.2 ± 0.9 | 6.0 ± 0.9 | 6.7 ± 0.8 | 0.016 |
| Fasting serum insulin (lU/mL) | 28.1 ± 26.1 | 25.1 ± 22 | 43.9 ± 39.1 | 0.130 |
| Ferritin (ng/mL) | 199.8 ± 180.2 | 210 ± 189.4 | 132 ± 73.2 | 0.032 |

The histologic features and differences in the study cohort classified by the advanced fibrosis status are presented in Table 1.2. Patients with advanced fibrosis were more likely to have more severe lobular and portal inflammation and ballooning than those without advanced fibrosis.

TABLE 1.2
Histological Features of patients with NAFLD by fibrosis status

| | | | Stage 0-2 | | Stage 3-4 | | |
|---|---|---|---|---|---|---|---|
| | | | Healthy, Moderate | | Advanced | | |
| | | Score/ | Fibrosis | | Fibrosis | | p-value |
| Histological Feature* | Definition | Code | N = 72 | | N = 14 | | (χ²) |
| Steatosis: | | | | | | | |
| Grade | Low- to medium-power | | | | | | |
| | evaluation of parenchymal | | | | | | |
| | involvement by steatosis | | | | | | |
| | <5% | 0 | 4 | (5.6%) | 1 | (7.1%) | |
| | 5%-33% | 1 | 25 | (34.7%) | 9 | (64.3%) | |
| | >33%-66% | 2 | 29 | (40.3%) | 1 | (7.1%) | |
| | >66% | 3 | 13 | (18.1%) | 2 | (14.3%) | 2.6e−14 |
| Inflammation: | | | | | | | |
| Lobular | Overall assessment of all | | | | | | |
| inflammation | inflammatory foci | | | | | | |
| | (no. foci per 200X field) | | | | | | |
| | No foci | 0 | 4 | (5.6%) | 0 | (0%) | |
| | <2 foci | 1 | 39 | (54.2%) | 2 | (14.3%) | |
| | 2-4 foci | 2 | 26 | (36.1%) | 9 | (64.3%) | |
| | >4 foci | 3 | 2 | (2.8%) | 0 | (0%) | 2.6e−14 |
| Portal | Assessed from low | | | | | | |
| Inflammation | magnification | | | | | | |
| | None | 0 | 15 | (20.8%) | 2 | (14.3%) | |
| | Mild | 1 | 42 | (58.3%) | 3 | (21.4%) | |
| | Greater than mild | 2 | 3 | (4.2%) | 4 | (28.6%) | 1.9e−11 |
| Liver cell injury: | | | | | | | |
| Ballooning‡ | None | 0 | 26 | (36.1%) | 1 | (7.1%) | |
| | Few balloon cells | 1 | 32 | (44.4%) | 4 | (28.6%) | |
| | Many cells/prominent | 2 | 8 | (11.1%) | 7 | (50%) | 4.2e−06 |
| | ballooning | | | | | | |

*Determination of histological features from centrally reviewed biopsy using the NASH Clinical Research Network Scoring System (Kleiner et al, Hep 2005)
‡Ballooning classification: few indicates rare but definite ballooned hepatocytes, as well as cases that are diagnostically borderline
§ The “None to rare” category is meant to alleviate the need for time-consuming searches for rare examples or deliberation over diagnostically borderline changes. If the feature is identified after a reasonable search, it should be coded as “many.”

Stool samples from the individuals in Group 1 and Group 2 were obtained and the gut microbiome compositions of the samples were determined using whole-genome shotgun sequencing of extracted DNA as follows.

DNA extraction. A 3-mL volume of lysis buffer (20 mM Tris-HCl pH 8.0, 2 mM Sodium EDTA 1.2% Triton X-100) was added to 0.5 grams of stool sample, and sample was vortexed until homogenized. A 1.2 mL volume of homogenized sample and 15 μl of Proteinase K (Sigma Aldrich, PN. P2308) enzyme was aliquoted to a 1.5 mL tube with garnet beads (Mo Bio PN. 12830-50-BT). Bead tubes were then incubated at 65° C. for 10 minutes and then 95° C. for 15 minutes. Tubes were then placed in a Vortex Genie 2 to perform bead beating for 15 minutes and the sample subsequently spun in an Eppendorf Centrifuge 5424. 800 μL of supernatant was then transferred to a deep well block and DNA extracted and purified using a Chemagic MSM I (Perkin Elmer) following the manufacturer's protocol. Zymo Onestep Inhibitor Removal kit was then performed following manufacturer's instructions (Zymo Research PN. D6035). DNA samples were then quantified using Quant-iT on an Eppendorf AF2200 plate reader.

Library Preparation and Sequencing. Nextera XT libraries were prepared manually following the manufacturer's protocol (Illumina, PN. 15031942). Briefly, samples were normalized to 0.2 ng/μL DNA material per library using a Quant-iT picogreen assay system (Life Technologies, PN. Q33120) on an AF2200 plate reader (Eppendorf), then fragmented and tagged via tagmentation. Amplification was performed by Veriti 96 well PCR (Applied Biosystems) followed by AMPure XP bead cleanup (Beckman Coulter, PN. A63880). Fragment size for all libraries were measured using a Labchip GX Touch Hi Sens. Sequencing was performed on an Illumina HiSeq 2500 using SBS kit V4 chemistry.

Metagenomic data annotation: Microbiome sequence data were processed as previously described in Jones, M. B. et al., Proc Natl Acad Sci USA, 112(45):14024-14029 (2015). The annotation pipeline generated relative genome abundance estimates of the constituent microbes in the samples and relative abundances of protein families (COGs, Pfams, TIGRFAMs, and EC). As part of the annotation process, data from each metagenomic sample was also assembled to generate contig assemblies. Contigs were assigned taxonomy and organized into species bins. The annotation information was then used to carry out metabolic reconstructions of the assembled species using Pathway Tools (Karp, P. D., et al. Bioinformatics, 18 Suppl 1:S225-S232. (2002)). ORFs were generated from assembled data and singleton reads using MetaGene. The relative abundance of a protein family is sum of ORF abundances. The relative abundance of a pathway is defined to be sum of relative abundances of all species where that pathway was reconstructed.

Random Forest: The Random Forest algorithm was used for two purposes: 1) to model microbial signatures of liver fibrosis; and 2) to select important species that may contribute most to the progression of liver fibrosis. Species relative abundances and patient data, also referred to as features, were analyzed using the Random Forest package in R (Breiman, L. Machine learning 45, no. 1, 5-32 (2001); Liaw, A. et al., R news 2, no. 3, 18-22 (2002)). A forest is trained by supervised learning in which each tree in the forest finds an ideal split for a set of randomly chosen features such that the predicted outcome of each sample is the same as the expected outcome. The data partition found by every tree in a forest is used to vote on a predicted overall outcome of the samples. The voting strategy of Random Forest is documented in the literature to avoid the over fitting of data due to the random sampling of features by each tree. Using every tree to vote on an outcome prevents any single tree that may have memorized the data from having a dominant prediction.

Outcomes are disease or no disease. AUC or Area Under the Receiver-Operator Curve measured the accuracy of trained forests. AUC is a widely used estimator of true positive and false positive prediction rates. Variable or species importance lists from those forests with the highest AUCs were selected for further analysis.

Training Data: The dataset consisted of sample diversity, sample richness, and the relative genome abundances of species detected in 86 stool samples collected from patients in a Registry Cohort. Age, Gender, Race, and BMI of each patient were also included in the training set. For this study, individuals were categorized into two groups based on the severity of fibrosis. The first group (Group 1) of individuals had no fibrosis (Stage 0) or mild/moderate fibrosis (Stages 1 and 2). A second group (Group 2) consisted of those patients whose livers were biopsied to confirm their advanced stages of fibrosis (Stages 3 and 4). Most patients (72) were in Group 1 and 14 patients were part of Group 2. Patient profiles Age, Race, BMI, and Gender with respect to the different stages of fibrosis are shown in Table 1.1. To reduce the level of noise that may be present in the relative abundance data, abundances that were less than 10⁻⁴ were set to zero and a species must be present in more than 70% of the patient stool samples.

Hierarchical Clustering: To reduce the effect that correlated data may have on training the abundance data was further filtered by hierarchical clustering. The cor function in R was used to calculate the Spearman correlation coefficients from species abundance data. The correlation matrix was converted to a dissimilarity matrix before using the hclust function for a complete linkage clustering of the dissimilarity matrix. The cor and hclust functions are part of the R STATS package. The resulting tree from the clustering was cut at a height of 0.1 and the species that was the closest to all other species within a cluster was chosen as a representative species from that cluster. When this procedure was applied to the initial set of 152 species, it resulted in 136 representative species, which were subsequently used for the training phase. A list of the species clusters generated is shown in Table 1.3.

TABLE 1.3

[symbol]

_{—}sp._{—}SS2.1
Lachnospiraceae_bacterium_5_1_63FAA
Lachnospiraceae_bacterium_CAG.25
Anaerostipes_hadrus
butyrate.producing_bacterium_SSC.2 [symbol]
_{—}sp._{—}D2
Lachnospiraceae_bacterium_5_1_63FAA
Bacteroides_uniformis_CAG.3

[symbol]

Lachnospiraceae_bacterium_5_1_63FAA
Bacteroides_vulgatus_CAG.6

[symbol]

Dorea_longicatena_CAG.42

[symbol]

Eubacterium_hallii_CAG.12

[symbol]

Eubacterium_rectale_CAG.36 [symbol]
_{—}3_{—}1_{—}46FAA
Lachnospiraceae_bacterium_1_1_57FAA
Lachnospiraceae_bacterium_8_1_57FAA [symbol]
_{—}2_{—}1_{—}58FAA
Ruminococcus_gnavus [symbol]
_{—}sp._{—}1.3
Oscillibacter_sp._CAG.155 [symbol]
_{—}sp._{—}5_{—}1_{—}39BFAA
Ruminococcus_sp._CAG.9

[symbol]

Streptococcus_salivarius
*Species in bold have correlated abundances to representative species

Initial Training of Random Forests: A series of steps to train a Random Forest with the best overall accuracy of classification was developed, reported as AUC. 300 forests were trained, containing 1001 trees each, with the relative genome abundances of species that passed abundance and prevalence filtering as previously described. In addition, the Shannon Diversity Index and richness of each sample, and the age, BMI, gender, and race of each patient were also included in the training set. Due to the small number of patients in Group 2 in comparison to Group 1, training was done with stratified sampling in which features from an equal number of samples from each group were randomly sampled and used to train each tree. A trained forest produces a variable importance list based on mean decrease in Gini index. For the dataset the variable importance list is a list of species, sample indices, or patient measurements that contributed most to the correct classification or the correct group assignment of every sample. The species importance list from the forest with the highest AUC was selected for Iterative Feature Elimination (described below). To determine the significance of the performance of the trained forest in this step, a Monte-Carlo simulation was used in which an additional 10,000 forests using permuted class values were trained.

Iterative Feature Elimination (IFE) and Forest/Feature Selection: Features (species, sample indices, and patient data) from the feature importance list described in the previous section were iteratively eliminated to find a set of features that trains a forest with the highest overall accuracy of sample classification. The feature importance list was ordered from highest to lowest Mean Decrease in Gini index and the least important species was removed. A random forest was trained with the remaining features in the feature importance list and an AUC is calculated. Removing least important features, training a forest with the remaining features, and calculating an AUC was continued until all of the features from the importance list were removed. The features used to train a forest with the highest AUC were used as the final feature importance list. In the case where there are two or more forests with the highest AUC, the forest with the largest number of features was chosen. The species that trained the forest with the highest AUC after the feature elimination step are reported in the final model.

Significance of Species Selection: To determine the significance of the final species importance list, a Monte-Carlo simulation approach was used in which a null distribution of AUCs from forests trained on randomly chosen features was created. The number of randomly chosen features is the same number of features found by the Iterative Feature Elimination step as described in the previous section. AUCs were calculated for 10,000 forests trained on randomly selected features and used to form a null distribution from which to compare against the significance of the top features selected by iterative feature elimination (IFE features). A p-value associated with the IFE features is the fraction of times that the AUC of forests trained on randomly selected sets of features were higher than the AUC of the forest trained by the IFE features.

Linear Support Vector Machine: Linear support vector machine (linear SVM) is used for two procedures: (1) feature selection, i.e. selection of important patient data and microbial species, and (2) classifier training with selected features. Feature selection is done with L1 norm regularization and classifier training is done with L2 norm regularization. Dataset used for linear SVM is the same as for Random Forest classification. Group 1 with mild/moderate fibrosis was assigned with class label “−1” and Group 2 with advanced fibrosis was assigned with class label “1”. Feature set consists of patient data, including sex, age, BMI, race (White, Asian, Hispanic) and referred to as metadata, and microbial species present in more than 70% of the 86 samples in the registry cohort. Linear SVM module sklearn.svm. Linear SVC from Python was applied and a grid search for penalty parameter C in range 2⁻⁵ to 2⁵ was performed to pick the best estimator parameters. Stratified 2-fold cross-validation was used to configure training and testing datasets. ROC-AUC was used as the scoring method to evaluate accuracy of the classifier on testing dataset.

Feature Selection with L1 Norm: Linear SVM with L1 norm penalty was used for feature selection on feature set containing numeric metadata (age, BMI), binary metadata (female, Hispanic, Asian, White), and log-transformed relative abundances of 152 microbial species. 24 features are selected with non-zero coefficients under L1 regularization, including 4 metadata (age, female, Asian, Hispanic) and 20 microbial species. These selected features were used as new feature set for the next step training of linear SVM classifier.

Significance of SVM Selected Feature Set: To determine the significance of the set of selected features, a null distribution of ROC-AUC scores was created in the following procedure: (1) randomly choose 20 microbial species from 152 species list, (2) combine 4 metadata and 20 random microbial species as a new feature set, (3) train linear SVM with L2 norm using the new feature set, (4) calculate AUCs using stratified 2-fold cross-validation, (5) repeat random species selection 10,000 times to form the null distribution. P-value was obtained by comparing AUC of the selected feature set to the null distribution (data not shown).

Concordance of RF and SVM models on the biopsy proven NAFLD cohort: The trained SVM selected 18 species as predictors (Table 3) and 12 of those species overlapped with the species selected by the Random Forest method (identified in Table 3 by bold font and set forth in Table 4).

Statistical test for difference in relative abundance: Wilcoxon Rank Sum test was used to assess differential abundance. Multiple test correction was used when appropriate and tests were controlled for false discovery rate at significance level of 0.05.

Age-Balanced Dataset: All patients in the advanced stages (stages 3 and 4) of fibrosis from the biopsy-proven registry cohort (Cohort A, 86 patients) were 60 years of age or older. The skew in age was not as extreme for patients in Group 1 such that a wider range of ages was observed for patients with either Stage 0, 1, or 2. To address the observed skew in age for patients with advanced fibrosis, a second cohort, referred to as Cohort B, of patients that are all 60 years or older from multiple cohorts was created. The 49 patients in Cohort B consist of 31 patients from Cohort A, 16 healthy patients from a cohort of twins (single twin from each pair), and two biopsy-proven cirrhotic patients from a familial cirrhosis study.

Metabolite Profiles: Metabolites were identified using Metabolon's mass spectrometry based metabolic profiling of serum samples (Guo, L. et al., Proc. Natl. Acad. Sci.,112(35):E4901-E4910 (2015)). Serum samples from 56 individuals (50 from Group 1 and 6 from Group 2) were used to generate metabolites profiles. Significant metabolites and their p-values are shown in Table 1.4.

TABLE 1.4

| Metabolite | Log₂(G2/G1) | p-value | Accession |
|---|---|---|---|
| malate | 0.722 | 0.0004455 | MAL |
| α-ketoglutarate | 1.04 | 0.0005091 | HMDB00208 |
| succinate | 0.319 | 0.0008286 | HMDB00254 |
| glutamine | 0.323 | 0.001388 | GLN |
| lactate | 0.346 | 0.001954 | L-LACTATE |
| hypoxanthine | −0.964 | 0.002676 | HYPOXANTHINE |
| fumarate | 0.502 | 0.003898 | FUM |
| serine | 0.35 | 0.003963 | SER |
| inosine | −3.19 | 0.004279 | INOSINE |
| α-ketobutyrate | 0.733 | 0.00546 | 2-OXOBUTANOATE |
| glutamate | 0.925 | 0.005976 | GLT |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method to detect liver fibrosis or for the differential diagnosis of type of non-alcoholic fatty liver disease (NAFLD) in a subject, comprising:
analyzing a biological sample from a subject to determine an intestinal microbiome signature for the subject; and
inspecting the intestinal microbiome signature relative to a reference intestinal microbiome signature to detect presence or absence of liver fibrosis; or
inspecting the intestinal microbiome signature to determine whether at least n bacterial species identified in Table 2 is present or absent in the signature, where n is at least 2, wherein presence or absence of the at least n bacterial species identified in Table 2 in the intestinal microbiome signature indicates nonalcoholic steatohepatitis (NASH).

2. The method of claim 1, wherein analyzing comprising applying the biological sample to a test panel that detects at least n bacterial species identified in Table 2, where n is at least 2.

3. The method of claim 2, wherein analyzing further comprises defining an intestinal microbiome signature according to the presence or absence of the at least n bacterial species identified in Table 2.

4. The method of claim 1, wherein the reference intestinal microbiome signature is obtained from a population of subjects without liver fibrosis.

5. The method of claim 1, wherein the reference intestinal microbiome signature is obtained from a population of subjects with liver fibrosis.

6. The method of claim 5, wherein the population of subjects have advanced liver fibrosis.

7. The method of claim 1, wherein a relative abundance of bacterial species in the intestinal microbiome signature is determined based on a median abundance of each bacterial species in the intestinal microbiome signature relative to a median abundance of each bacterial species in the reference intestinal microbiome signature.

8. The method of claim 1, wherein analyzing a biological sample comprises analyzing a sample selected from the group consisting of a stool sample, an intestinal mucosal sample and a sample of the intestinal contents.

9. The method of claim 1 wherein based on the inspecting, a stage of liver fibrosis is determined.

10. The method of claim 9, wherein based on the inspecting a stage of advanced fibrosis is determined.

11.-12. (canceled)

13. The method of claim 1, wherein n is at least 8 and is comprised of the bacterial species in Group A (Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae.

14. The method of claim 13, wherein the intestinal microbiome signature comprised of the Group A bacterial species have a relative abundance of each bacterial species in the signature at least two-fold higher than a relative abundance of the Group A bacterial species in a reference intestinal microbiome signature.

15. The method of claim 13, wherein n is at least 9 and additionally comprises one or more of the bacterial species in Group B (Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alistipes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG: 83, Dorea longicatena, Firmicutes bacterium CAG: 129, Ruminococcus obeum CAG:39, Blautia sp. CAG:37, Eubacterium rectale, Firmicutes bacterium CAG: 176, Firmicutes bacterium CAG: 110, and Holdemania filiformis).

16. The method of claim 15, wherein the Group B bacterial species in the intestinal microbiome signature have a relative abundance at least two-fold lower than a relative abundance of the Group B bacterial species in a reference intestinal microbiome signature.

17. The method of claim 1, wherein n is comprised of the bacterial species in Group C (gathobacter rectalis (Eubacterium rectale), Blautia sp KLE 1732, Roseburia inulinivorans, Oscillibacter (genus), Eubacterium ramulus, and Blautia sp. GD8).

18. The method of claim 17, wherein the Group C bacterial species in the intestinal microbiome signature have a relative abundance at least two-fold lower than a relative abundance of the Group C bacterial species in a reference intestinal microbiome signature.

19. The method of claim 1, wherein analyzing comprises analyzing the biological sample using a microarray comprising nucleic acid sequences with binding affinity for one or more bacterial species set forth in Table 2.

20-27. (canceled)

28. The method of claim 1, wherein analyzing comprises analyzing using a method selected from microscopy, metabolite identification, Gram staining, flow cytometry, immunological assays, and culture-based assays.

29. A method for the differential diagnosis of the type of non-alcoholic fatty liver disease (NAFLD) in a subject, comprising:
determining an intestinal microbiome signature of the subject, wherein a diagnosis of stage 3-4 fibrosis is indicated by one or more of the following criterion:
(a) the intestinal microbiome signature of the subject having a relative abundance of a bacterial species that is at least two-fold higher than the relative abundance of bacterial species in a reference intestinal microbiome signature, wherein the bacterial species is selected from the group consisting of Dorea sp. CAG:317, Bacteroides cellulosilyticus, Bacteroides finegoldii, Bacteroides dorei, Streptococcus parasanguinis, Clostridium symbiosum, Clostridium sp. 7_3_54FAA, and Clostridium bolteae (Group A);
(b) the intestinal microbiome signature of the subject having a relative abundance of a bacterial species that is at least two-fold lower than the relative abundance of bacterial species in a reference intestinal microbiome signature, wherein the bacterial species is selected from the group consisting of Subdoligranulum sp. 4_3_54A2FAA, Bacteroides sp. 1_1_30, Faecalibacterium sp. CAG:82, Clostridium sp. L2-50, Blautia sp. KLE 1732, Clostridium sp. CAG:43, Firmicutes bacterium CAG:56, Ruminococcus sp. CAG:17, Ruminococcus obeum, Alishpes putredinis, Roseburia inulinivorans, Ruminococcus sp. CAG:90, Bacteroides pectinophilus, Roseburia intestinalis, Coprococcus comes, Oscillibacter sp. CAG:241, Firmicutes bacterium CAG:83, Dorea longicatena, Firmicutes bacterium CAG:129, Ruminococcus obeum CAG:39, Blautia sp. CAG:37, Eubacterium rectale, Firmicutes bacterium CAG:176, Firmicutes bacterium CAG:110, and Holdemania filiformis (Group B);
(c) the intestinal microbiome signature of the subject having a relative abundance of a bacterial species that is at least about two-fold lower than the relative abundance of the bacterial species in a reference intestinal microbiome signature, wherein the bacterial species is selected from the group consisting of Agathobacter rectalis (Eubacterium rectale), Blautia sp. KLE 1732, Roseburia inulinivorans, Oscillibacter (genus), Eubacterium ramulus, and Blautia sp. GD8 (Group C); and
(d) the intestinal microbiome signature of the subject having (i) a relative abundance of at least two bacterial species in Table 2 that is at least two-fold higher than the relative abundance of the same two bacterial species in a reference intestinal microbiome signature and (ii) a relative abundance of at least two bacterial species in Table 2 that is at least two-fold lower than the relative abundance of the same two bacterial species in a reference intestinal microbiome signature, and wherein the sum of the mean decrease in Gini index of the at least two bacterial species in (i) and of the at least two bacterial species in (ii) is greater than 0.5.

30. The method of claim 29, wherein the diagnosis of stage 3-4 fibrosis is indicated by both (a) and (b), by both (a) and (c), by both (a) and (d), by both (b) and (c), or by both (b) and (d).

31-35. (canceled)

35. A substantially non-invasive method for assessing risk of progression to liver cirrhosis in a subject having an intestinal microbiome signature and diagnosed with non-alcoholic fatty liver disease (NAFLD), comprising:
determining whether at least n bacterial species identified in Table 2 is present in the intestinal microbiome signature of the subject, where n is at least two, wherein presence of the at least n bacterial species identified in Table 2 in the intestinal microbiome signature indicates risk of progression to liver cirrhosis; or
determining from the intestinal microbiome signature of the subject (i) a relative abundance of at least two bacterial species in Table 2 that is at least two-fold higher than the relative abundance of the same two bacterial species in a reference intestinal microbiome signature and (ii) a relative abundance of at least two bacterial species in Table 2 that is at least two-fold lower than the relative abundance of the same two bacterial species in a reference intestinal microbiome signature, and (iii) a mean decrease in Gini index of the at least two bacterial species in (i) and of the at least two bacterial species in (ii), wherein if the sum of the mean decrease in Gini index is greater than 0.5 risk of progression to liver cirrhosis is indicated.

36-38. (canceled)
